(19) United States
(12) Patent Application Publication
(10) Pub. No.: US 2020/0108077 A1
(43) Pub. Date: Apr. 9, 2020

(54) GYMNEMA BASED ORAL COMPOSITION

(71) Applicant: NU Brands, Los Angeles, CA (US)

(72) Inventors: Sean Byrne, Los Angeles, CA (US); Harley Pasternak, Los Ageles, CA (US)

(21) Appl. No.: 16/153,569

(22) Filed: Oct. 5, 2018

(51) Int. Cl. A61K 31/56; A61K 9/00; A61K 36/24; A61K 33/24; A61K 9/20

(52) U.S. Cl. CPC A61K 31/56; A61K 9/0053; A61K 36/24; A61K 9/0056; A61K 9/2018; A61K 9/2013; A61K 9/205; A61K 33/24

(57) ABSTRACT

An oral composition that delivers a dose of an active ingredient, e.g., gymnemic acid (GA), directly to the sweetness taste receptors of an oral cavity, which blocks the sense of sweetness and thereby produces appetite control. The oral composition contains a sweet-suppressing agent as an active ingredient (e.g., GA), a sweetening agent (e.g., inulin), and a flavor (e.g., spearmint or cinnamon). The oral composition can include an appetite-suppressing agent (e.g., chromium.) The sweet-suppressing agent can interact with taste receptors in an oral cavity of a consumer to temporarily suppress the taste of sweetness. The sweetening agent can decrease the bitterness taste of the sweet-suppressing agent. The oral composition can be in any dosage form, e.g., an orally disintegrating tablet, an orally dispersible tablet, a troche, a candy, a jelly, a gum, an edible film or a strip, a wafer, a drop, an oral spray, a liquid, or powder.

### BACKGROUND

The present invention relates to a diet for suppressing the absorption of saccharides from the intestines.

As it is called an age of satiation, in recent years, people have come to enjoy the table full of variety and delicacies, and while the intake of sugars, starches, and the like becomes excessive, the quantity of people's exercise has decreased, so that people have become corpulent from their early childhood and are susceptible to various geriatric diseases such as diabetes mellitus.

As means of preventing one from having corpulence that will induce geriatric diseases, diet therapies wherein the intake of starches and the like is restricted are followed, but these therapies are tiresome and require one's strong determination. Therefore, artificial sweetening that are not absorbed into the intestines have been developed and used, but these only decrease the amount of sugars and have no relation with the absorption of saccharides originated from carbohydrates, so that they do not provide a complete solution.

Diets that comprise, as raw material, Gymnema sylvestre produced in India, having an effect for suppressing the absorption of saccharides and intended for the prevention of corpulence and for the suppression of the increase of blood sugar, have been suggested.

The extract of Gymnema Sylvestre (GS extract) which is a perennial plant of Asclepiadaceae growing naturally in India, Southeast Asia and China has been known as having the function to suppress the sensibility to sweetness from old times. Although the physiological mechanism of the gymnemic acid (GA) contained in the GS extract is not always apparent, GA has recently attracted attention because it has the pharmacological effect of inhibiting the absorption of sugar in the human digestive system. There have been thus many proposals of use as a medicine for diabetes and as health food, and practical applications thereof. The present day is called the age of satiation and overeating, and brings about increases in the degree of obesity due to excessive caloric intake and lack of exercise, thereby causing increases in the number of diseases of adult people. There is also the problem of apositia of young women caused by excessive diet. GS extract is strong in bitterness and astringency and act on the sweetness receptor of taste cells in the mouth, thereby exhibiting a sweetness sensation suppressing effect that prevents the combination of a sweetener with the sweetness receptor.

Although the GS extract has the effect of inhibiting sugar absorption, it has the problem of strong peculiar bitterness, and the problem of making the taste buds which is sensible to sweetness in the mouth insensible to the sweetness of sweat food due to the anesthesia thereof. There is thus no practical application in which the GS extract is contained in food for tasting sweetness. Namely, since sweetness of food is generally an important factor for a good taste, the insensibility to sweetness causes a bad taste, resulting in unwillingness to eat.

There are thus conventional proposals for providing sweet food as health food which permits a taste of sweetness in the mouth, and which is effective for preventing obesity by inhibiting absorption in the digestive system such as the intestinal tract. For example, health food of the type in which the GS extract is involved in cyclodextrin is proposed, and is actually commercially available.

In order to decrease caloric intake, efforts to decrease the intake of sugar and lipid, and-to take low-calorie substitute food are extensively made. However, since it is necessary for preventing obesity and limiting caloric intake to exert the efforts at every meal every day, a strong will and power of execution are required for actually exerting the efforts, thereby causing difficulties. Such self-control rather frequently causes new frustration.

### DETAILED DESCRIPTION

An object of the present invention is to provide an oral composition that delivers a relatively small dose of an active ingredient, e.g., gymnemic acid (GA), directly to the sweetness taste receptors of an oral cavity, which blocks the sense of sweetness and thereby produces appetite control. A person or subject (“consumer”) consuming the oral composition may no longer be able to experience the sweet taste of what they are eating, drinking or introducing into the oral cavity, rendering most foods, drinks, and other substances unpalatable. Thus, the oral composition allows the consumer to address their cravings for sweet foods immediately. Moreover, through extended use of the oral composition, the consumer of the oral composition may modify their behavior because they no longer associate a pleasant feeling with foods that previously tasted sweet. Thus, the oral composition also aids in reducing sugar consumption and calorie intake by a consumer. The oral composition can also aid in weight loss through the appetite control and/or reduced calorie and/or sugar intake.

The oral composition contains a sweet-suppressing agent as an active ingredient (e.g., GA), a sweetening agent (e.g., inulin), and a flavor (e.g., spearmint or cinnamon). In some embodiments, the oral composition includes an appetite-suppressing agent (e.g., chromium). The sweet-suppressing agent can interact with taste receptors in an oral cavity of the consumer to temporarily suppress the taste of sweetness. The sweetening agent can decrease the bitterness taste of the sweet-suppressing agent to make the oral composition more palatable. The flavor can improve palatability of the oral composition.

The oral composition can be in any dosage form intended to be held in the oral cavity for sufficient time to allow the active ingredients to contact the tissues of the oral cavity to block the sweetness taste receptors in the oral cavity, thereby decreasing the sensitivity of sweetness receptors to a taste of sweetness. Examples of such dosage forms include a tablet cake, a granule oral refrigerant, a lozenge, an orally disintegrating tablet, an orally dispersible tablet, a troche, a hard candy, a soft candy, a jelly, a gum, an edible film, an orally dissolvable film, a wafer, a drop, an oral spray, a liquid, a powder and combinations thereof. The disintegration time of the orally disintegrating tablet may be in the range of from about 30 seconds to about 5 minutes. At least some dosage forms the oral composition, such as an orally disintegrating tablet, can have a filler-binder, base, or an excipient (e.g., sorbitol) and an anti-caking agent (e.g., Magnesium Stearate). The oral cavity can include the tongue, inside of the mouth, the buccal cavity, under the tongue, and the like. Sweetness taste receptors are located in taste bud cells primarily on the tongue but are also found on the soft palate and other regions of the oral cavity.

In some embodiments, the oral composition includes GA as the sweet-suppressing agent, inulin as the sweetening agent, spearmint or cinnamon as the flavor, and sorbitol as the filler-binder. The GA is present in the oral composition in any amount effective to partially or completely block the sensation of sweet taste in a subject for a period of time. Example of such an amount includes, but is not limited to, from about 0.3 to about 130 mg GA, preferably 35 mg. In some embodiments, a dose of about 35 mg of gymnemic acid delivered in an orally disintegrating tablet form has the ability to block the sense of sweetness for a period of up to 2 hours, thus discouraging the consumption of food and controlling the appetite. The oral composition according can include Gymnema sylvestre herb or, preferably, an extract of Gymnema sylvestre herb designed to maximize the quantity of gymnemic acid it contains. The oral composition can also include from about 3 mg to about 10 mg of the sweetening agent, preferably 5 mg, and one or more flavors per unit amount of the composition taken at a time. In some embodiments, the oral composition includes from about 200 mcg to about 1000 mcg of the appetite-suppressing agent, preferably 600 mcg.

In some embodiments, the oral composition is an orally disintegrating tablet containing 35 mg of GA, 5 mg of inulin, spearmint and/or cinnamon flavors, and sorbitol as the filler-binder. In embodiments of the oral composition that have chromium (appetite-suppressing agent), chromium may be present in an amount of 600 mcg. Note that the oral composition is not limited to the above ingredients; it may have other ingredients as well. Below table lists some of the ingredients of the oral composition prepared as an orally disintegrating tablet.

TABLE 1
Tablet

| Ingredients | |
|---|---|
| Gymnema Extract/Gymnemic Acid | 35 mg |
| Inulin | 5 mg |
| Spearmint or cinnamon flavor | |
| Sorbitol | |
| Magnesium Stearate | |

Gymnema sylvestre (GS), commonly called Gurmar, is well known in the art as a “sugar blocker.” Gymnema sylvestre contains the bitter principle GA, which is well known for its ability to bind to the sweetness taste receptors on the tongue and to sugar absorption sites in the gut. The bitter taste of GA can limit the use of GS in the above dosage forms. GA is known to selectively bind to the sweetness receptors of the tongue and eliminate the sense of sweetness for a specified duration, e.g., 1 to 2 hours. In some embodiments, a sweetening agent, preferably a natural sweetening agent, is included in the oral composition to decrease or mask the bitterness of GA and make the oral composition more palatable. Examples of natural sweetening agent include inulin and chromium. Inulin is also a good source of dietary fiber and aids in weight loss. In some embodiments, other sweetening agents that aid in weight loss can also be considered.

One or more of various flavors can also be added to the oral composition for improving palatability. For example, the oral composition can include a spearmint or cinnamon flavor. Spearmint is generally a sweet flavor imparting a cool sensation to the mouth. Spearmint is either naturally or artificially created to taste like the oil of the herbaceous Mentha spicata (spearmint) plant.

The oral composition is useful in preventing obesity among human beings, e.g., for a person such as a diabetic who must diet and limit calorie, and for a person with hyperphagia from overeating and eating between meals. Although liquors such as whisky, beer and sake are high-calorie drinks, and overdrinking thereof of course causes obesity, since the intake of the oral composition prevents overdrinking of such alcoholic drinks, the oral composition is effective for preventing obesity. The oral composition is also effective for helping a person who is apt to overdrink alcohol and a person of alcohol dependence to be temperate in drinking and abstain from drinking.

The oral composition of the present invention may contain either purified GA or unpurified GS extract. The GS extract can be obtained by extraction of GS dry leaves with water or an aqueous solution containing alcohol. The unpurified GS extract represents the product obtained as about 250 g solid by a method in which an aqueous solution containing 30% ethanol is added to 1 kg of dry GS leaves at an amount ratio of 11:1, followed by extraction at 80 degree Celsius for 2 hours, the leaves are then filtered off, and the filtrate is concentrated and then powdered by spray drying. The amount of the GS extract corresponding to GA is 5 to 500 mg, preferably 10 to 60 mg, per unit amount of the oral composition taken at a time. Although GA is known as a compound having triterpene as a skeleton and gluconic acid, it is a glycoside having at least 20 homologues and a quantitative method has not been established as yet. GA is generally defined as being extracted from the GS extract precipitated at pH 3 or less, with a 30 to 70% alcohol solution. Further, in some cases instead of the GS extract, GS dry leaves finely crushed 100 mesh may be added in an amount 2 to 4 times larger than the extract.

### EXAMPLES

Although the present invention is described below with reference to examples, the present invention is not limited to the examples. The panelists A-G were people in the age group of 20-50 years. Formulation Procedure used to make the oral composition in a tablet dosage form having at least the ingredients from table 1:
Blend Flavor, Ethanol & Gymnema extract in a beaker until dissolved
Add entire portion directly to sorbitol premix, shake for 3-4 minutes
Add Inulin and shake
Add Magnesium Stearate
Press formulation into tablets

### Example 1

Sweetness Inhibition of Gymnemic Acid (35 mg Dosage) in an orally disintegrating tablet form. The oral composition was evaluated to quantify the duration of its sweet taste inhibition. Results obtained are summarized in Table 2. The results were consistent among most of the panelists.

TABLE 2

| Time (minutes) | Observations |
|---|---|
| 0 | No sweetness; strong bitterness. |
| 15 | No sweetness; medium bitterness. |
| 30 | No sweetness; weak/medium bitterness. |
| 45 | Faint sweetness; weak bitterness. |
| 60 | Faint sweetness; faint bitterness. |
| 75 | Faint sweetness; faint bitterness. |
| 90 | Weak sweetness; no bitterness. |
| 105 | Weak sweetness; no bitterness. |
| 120 | Medium to strong sweetness; no bitterness. |

### Example 2

Taste of food after consuming the oral composition in an orally disintegrating tablet form. Results obtained are summarized in Table 3. The results shown in Table 3 revealed that (a) most of the panelists evaluated all foods to be unsavory, and had different tastes from ordinary tastes, and (b) all panelists had no sweet taste of sweet food such as coffee (with sugar), doughnut, milk chocolate and shortcake, and did not want to eat such types of food. The loss of the sensibility to sweetness continued for up to 120 minutes.

TABLE 3

| | Sugared | | Milk | |
|---|---|---|---|---|
| Panelist | Coffee | Doughnut | Chocolate | Short-Cake |
| A | Intastable; | unsavory; | Uneatable; | unsavory; |
| | not sweet; | light taste | unsavory; | light taste |
| B | not sweet; | not sweet; | not sweet; | not sweet; |
| | unsavory; | unsavory; | unsavory; | |
| C | Only bitter | Light taste; | unsavory; | unsavory; |
| | | intastable | light taste | light taste |
| D | Only bitter | Light taste; | unsavory; | unsavory; |
| | | intastable | | |
| E | Not sweet | unsavory; | unsavory; | Light taste; |
| | | intastable | | intastable |
| F | Light taste | intastable | unsavory; | Light taste |
| G | Only bitter; | unsavory; | uneatable; | unsavory; |
| | intastable | flavorless | intastable | light taste |

### Example 3

Taste of food after consuming the oral composition in an orally disintegrating tablet form. After ten adult diabetics of 46 to 65 years old were let consume the oral composition before a meal or between meals, changes in the sense of taste and the subsequent behaviors were observed. The results obtained are shown in Table 5 below. As seen from the results, most of the patients changed in the sense of taste, and about a half, i.e., four patients, became to refrain from overeating and eating between meals.

TABLE 4

| | |
|---|---|
| Number of patients who did not enjoy meal | 6 |
| Number of patients who did not enjoy sweet cake | 9 |
| Number of patients who did not enjoy fruit | 7 |

As can be appreciated from the examples, consuming the oral composition containing GA can temporarily decrease the sensibility to sweetness, thereby decreasing the amount of eating between meals, a meal or drinking. The oral composition thus has the effect of preventing overeating and overdrinking and therefore, controlling appetite or aiding in weight loss.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. An oral composition in a specified dosage form, comprising:
gymnemic acid present in an amount of about 35 mg;
a sweetening agent including inulin;
a flavor; and
a filler-binder, wherein the oral composition is palatable when delivered to an oral cavity.

2. The oral composition of claim 1, wherein the gymnemic acid is present in a range of about 0.3 to about 130 mg.

3. (canceled)

4. The oral composition of claim 1, wherein the gymnemic acid is extracted from Gymnema sylvestre herb.

5. (canceled)

6. The oral composition of claim 5, wherein the inulin is present in the range of about 3 mg to about 10 mg.

7. The oral composition of claim 5, wherein the inulin is present in an amount of 5 mg.

8-11. (canceled)

12. The oral composition of claim 1, wherein the filler-binder is sorbitol.

13. The oral composition of claim 1 further comprising:
magnesium stearate as anti-caking agent.

14. The oral composition of claim 1, wherein the flavor is spearmint or cinnamon.

15. The oral composition of claim 1, wherein the dosage form is an orally disintegrating tablet.

16. An oral composition, comprising:
gymnemic acid present in an amount of about 35 mg;
inulin;
chromium;
spearmint or cinnamon flavor; and
sorbitol.

17. An oral composition, comprising:
gymnemic acid in an amount of 35 mg;
inulin in an amount of 5 mg;
spearmint or cinnamon flavor; and
sorbitol, wherein the oral composition is palatable as an orally disintegrating tablet.

18-20. (canceled)
